(19) United States
(12) Patent Application Publication
(10) Pub. No.: US 2019/0099572 A1
(43) Pub. Date: Apr. 4, 2019

(54) Airway Device for Placement in Upper Respiratory Tract

(71) Applicant: Richard Lenhardt, Elmhurst, IL (US)

(72) Inventor: Richard Lenhardt, Elmhurst, IL (US)

(21) Appl. No.: 16/151,955

(22) Filed: Oct. 4, 2018

Related U.S. Application Data
(60) Provisional application No. 62/568,258, filed on Oct. 4, 2017.

(51) Int. Cl. A61M 16/04; A61M 16/00

(52) U.S. Cl. CPC A61M 16/0488; A61M 16/0003; A61B 1/2676; A61M 2016/0413; A61M 16/0434; A61M 2016/0027

(57) ABSTRACT

An airway device for being inserted into an operative position in the upper respiratory tract extending from a patient's mouth to the patient's hypopharyngeal space is disclosed. The airway device forms a bore through which various medical and/or surgical implements may be inserted into the patient's airways. The airway device is contoured to approximately match the contour of the upper respiratory tract.

### CROSS-REFERENCE TO PRIOR APPLICATIONS

This application claims the benefit of provisional U.S. Patent Application No. 62/568,258, filed Oct. 4, 2017, the content of which is incorporated in its entirety herein.

### FIELD OF THE INVENTION

The present invention generally relates to an aid for placement in the upper respiratory tract to aid with a surgical procedure, such as the placement and performance of bronchoscopy or the placement of an endotracheal tube, to secure an artificial airway in patients with lung conditions, and more particularly, the device of the present disclosure functions as a conduit along a patient's upper respiratory tract from the mouth to the hypopharyngeal (between base of tongue and vocal cords) space. It can incorporate multiple features in various combinations depending on the needs of the clinical situation.

### BACKGROUND

Flexible bronchoscopy is a medical procedure that is used to diagnose and treat a large variety of lung conditions. The procedure was invented in the 1960s and has revolutionized the evaluation of patients with lung diseases. Flexible bronchoscopy enables access to small segments of a patient's lung. Prior to invention of the flexible bronchoscope a rigid tube, called a rigid bronchoscope, was used to access a patient's lung for diagnostic and therapeutic reasons. This rigid bronchoscope is a large tube that cannot, because of its large diameter, access small segments of a patient's lung.

The flexible bronchoscope enables visualization of lung abnormalities, obtain fluid samples and biopsies, and treat patients with certain lung conditions. Because the invention described in this document is to be used primarily as an aid for flexible bronchoscopy, the term “bronchoscopy” will refer to the flexible bronchoscope, though modifications of the invention could enable the invention to be used as an aid for the rigid bronchoscope, as well as many other types of medical and surgical devices.

During bronchoscopy a patient is anesthetized such that they do not feel anxiety, pain, or other discomforts and remain still with minimal movement enabling the procedure to proceed successfully. The inner lining of the human airway contains a variety of nerve endings that sense pain, excessive secretions, and foreign material. When pain, excessive secretions, or foreign material trigger these nerve endings, humans cough and this cough can be quite vigorous so as to expel the undesired material. Such a reflex is necessary to protect the human lung from suffocation and sustain life. Preparing humans for bronchoscopy and sustaining them through the procedure typically involves the use of multiple medications. Medications can be given topically as topical anesthetics, which numb the nerve endings in the inner lining of the airways. Intravenous medications typically used for bronchoscopy include sedatives, which prevent anxiety, and narcotics, which prevent pain.

Though some patients can undergo bronchoscopy using topical anesthetics alone, the vast majority of patients require intravenous medications. A side effect of these intravenous medications is slowing respirations, which at times can be unpredictable and precipitous, to the point of being dangerous to the patient. Underutilizing intravenous medications for bronchoscopy is hazardous as well, as the patient may feel anxiety or pain if undertreated.

### SUMMARY OF THE INVENTION

The present disclosure provides an airway device that forms a conduit that may be inserted and removed from a patient's mouth to the hypopharyngeal space, for example during a bronchoscopy or other similar medical procedure to a patient's upper respiratory tract. The conduit may then be used during the medical procedure to guide tools, keep the patient's respiratory tract open, deliver other substances to various locations along the respiratory tract, and carry other medical devices into the respiratory tract.

According to some aspects, an airway device is disclosed that provides a secure route for placement of a bronchoscope or an endotracheal tube. In some arrangements, the airway device may keep soft tissue out of the way during procedures, which may occlude the airway prohibiting ventilation. In some arrangements, the airway device may bridge the external aspect of the patient's mouth to the hypopharynx, which delivers medical instruments into the patient's trachea. In some arrangements, the airway device may contain an oxygen delivery channel to provide a secure route to the patient's hypopharynx that optimizes delivery of oxygen to lungs. In some arrangements, the airway device may contain one or more suction channel that enable removal of upper airway secretions. In some arrangements, the airway device may contain one or more dedicated anesthetic channels that enable anesthetization of the upper airway during insertion of the device. The working anesthetic channel could subdivide along the walls of the device such that the exit ports at the tip of the device are multiple. In some arrangements, the airway device may contain a working video channel that provides a channel for transmission of real time view of the tip of the device to an operator, including, for example, 3D vision. In some arrangements, the airway device may contain a pCO2 (carbon dioxide) or other partial pressure gas measurement detector. This detector may be placed at various positions along the device, including the hypopharyngeal part of device, and transmit the reading to the mouth end. In some arrangements, the airway device may be manufactured with or otherwise include a system that enables the radius of curvature of the device to be controlled during the procedure. In some arrangements, material of which the device is made may be flexible sufficiently that it will to conform to the patient's anatomy yet firm enough not to collapse in the patient's airway. The part of the device that bridges the patient's dentistry may be formed of material that is firm enough so as not to be compromised during inadvertent patient biting. In some arrangements, contour, shape, and material of device is manufactured and/or arranged so as to optimize comfort in a patient's airway. In some arrangements, the main is contoured such that the distal end rest directly above the glottis or tracheal opening instead resting directly above the opening into the esophagus when inserted in its operative position in the upper respiratory tract. In some arrangements, the airway device may be manufactured in different sizes and contours such that the device will fit humans of different ages, shapes, and sizes. In some arrangements, the airway device may be manufactured so that the far, or distal, end rests at any location from the patient's oral cavity to space just above the vocal cords in the hypopharynx. In some arrangements, the airway device may be manufactured with and/or include impregnated medications that anesthetize the patient's airway lining. In some arrangements, the airway device may be manufactured to contain and/or include a break away functionality to enhance removal after placement of an endotracheal tube. In some arrangements, the airway device may be manufactured with and/or include any number and combination of features mentioned above.

In some arrangements, an airway device for being inserted into an operative position in the upper respiratory tract extending from a patient's mouth to the patient's hypopharyngeal space. The airway device includes a main body having a hollow tubular shape forming a bore extending from a proximal end to a distal end, wherein the main body is formed of a semi-rigid material that has an at-rest contour that approximates a curve of the patient's airway. The main body may be contoured to have a variable curve along its longitudinal length. The curve of the contour may vary from a long radius at the proximal end to a short radius at the distal end. The main body may have a length that is at least as long as the upper respiratory tract extending from the patient's mouth to the patient's hypopharyngeal space. An oxygen delivery channel may be arranged to provide a secure route to the patient's hypopharynx that optimizes delivery of oxygen to lungs. A suction channel may be arranged to enable removal of upper airway secretions from the patient. An anesthetic channel may be arranged to enable anesthetization of the upper respiratory tract during insertion of the device into the upper respiratory tract. The anesthetic channel may include branches along the main body to form multiple exit ports at the distal end. A misting nozzle may be located at the distal end of the anesthetic channel that is arranged to expel the anesthetic drug in mist form. A video channel may be arranged to provide transmission of real time view of the distal end of the to an operator. A partial pressure gas measurement detector may be arranged to obtain measurements from the along the upper respiratory tract. A break-away feature may be disposed in a tube wall defining the main body. The break-away feature may be arranged in the tube wall such that, after placement of the airway device in the upper respiratory tract and a placement of a medical device along the bore, the tube wall can be separated to open at one or more radial sections through the walls of the airway device, whereby the airway device may be removed from the upper respiratory tract without removing the medical device. The main body may be composed of two or more substances along the device's length. For example, the proximal end of the main body may be composed of a substance resistant to biting, whereas the distal end and central portions of the main body are formed of a more flexible material to allow the main body to deform a small amount to accommodate the contours of the patient's upper respiratory tract. The main body may be impregnated with an anesthetic agent. An inflatable cuff may be disposed near the distal end of the main body, wherein the inflatable cuff is arranged to be inflated to seal the upper respiratory tract. An adjustment mechanism may be included and arranged to change the radius of the curve of the contour of the airway device so as to optimize positioning of the distal end of the main body in the patient's upper respiratory tract.

These and other aspects, arrangements, features, and/or technical effects will become apparent upon detailed inspection of the figures and the following description.

### BRIEF DESCRIPTION OF THE DRAWINGS

FIG. 1 is a partial cross-sectional view of an airway device of the present invention disposed in an operational position inside of a patient;

FIG. 2 is a side, or lateral, view of an airway device of the present invention;

FIG. 3 shows a hypopharyngeal end, or distal end, of an airway device of the present invention and with obtuse angle;

FIG. 4 is cross-sectional view of an airway device of the present invention showing oxygen and suction ports along the inside diameter of the main body;

FIG. 5 is a side, or lateral, view of an airway device of the present invention with multiple optional features shown;

FIG. 6 is a cross-sectional view of mouth end, or proximal end, of an airway device of the present invention with multiple options shown;

FIG. 7 is a cross-sectional view of the hypopharyngeal end, or distal end, of an airway device of the present invention with yet further multiple options;

FIG. 8 is a cross-sectional view of an airway device of the present invention with a break-away feature seen from the mouth end;

FIG. 9 is a side, or lateral, view of an airway device of the present invention with a wire and channel system for controlling the radius of the airway device; and

FIG. 10 is a cross-sectional view of an airway device of the present invention with a wire and channel system that controls the radius of the airway device.

### DETAILED DESCRIPTION

When a patient undergoing bronchoscopy has slowing respirations, the blood oxygen level falls and there are suboptimal ways to enhance the delivery of oxygen to the patient's lungs. In some arrangements, the device of the present disclosure is designed to optimally deliver oxygen to a patient's trachea (windpipe) during bronchoscopy. Intravenous medications used for bronchoscopy cause the patient's upper airway to relax and the airway may inadvertently and subsequently collapse. The patient may then require emergent intubation of the trachea (with an endotracheal tube) because loss of a patent airway from the tip of the lips to the vocal cords (which is the entrance to the lungs) would soon become fatal. Should the airway collapse, the device of the present disclosure, and variants of it, may assist in preventing this collapse to occur, and if it does, to mitigate the effect of the collapse.

The most common location for the upper airway to collapse related to side effects of intravenous medications administered during bronchoscopy is posterior (towards back) to the tongue. The device of the present disclosure, in contrast, preferably extends past this area such that should the patient's airway collapse the invention will stent open an artificial airway providing for a patent airway to be intact during the collapse. Additionally, during collapsing conditions, the space of the patient's airway immediately adjacent to the invention will be partly stented open because of the presence of a tubular structure of the device of the present disclosure.

A variation of the device of the present disclosure is to assist placement of an endotracheal tube. If the patient's airway or respiratory system is compromised during bronchoscopy such that artificial ventilation is necessary to prolong life, an endotracheal tube is placed. This tube extends from outside the lips into the trachea below the vocal cords and when attached to a mechanical ventilator, oxygen delivery, which is necessary for life, can be safely and completely provided. This variation of the device of the present disclosure—to assist placement of an endotracheal tube—can be used with or without bronchoscopy.

The device of the present disclosure is described as used in human patients. It can be modified to be used in animals as well.

Before describing the specific examples shown in the drawings, some general aspects, arrangements, and features of the disclosed airway device are provided. In some arrangements, the airway device may be placed in a patient's upper airway to assist insertion of a bronchoscope, an endotracheal tube, or other medical devices. The airway device may function as a conduit between outside the patient's mouth and the hypopharyngeal space. The airway device may be shaped and contoured such that it curves appropriately for insertion into the patient's upper airway. The contour and material of the airway device may be selected so as to optimize the patient's comfort. The material of the airway device may be manufactured to enable flexibility to adjust to a patient's airway and yet be rigid enough to maintain a open conduit in the patient's upper airway. In some arrangements, the main body is contoured along a variable radius curve such that the distal end rest directly above the glottis or tracheal opening instead resting directly above the opening into the esophagus when inserted in its operative position in the upper respiratory tract. The hypopharyngeal end, or distal end, of the airway device may be shaped such that it enhances the passage of the device into a patient's upper airway. The airway device may be manufactured in various shapes and sizes to fit to patients of different shapes and sizes. The airway device may include one or more channels configured to enable delivery of oxygen or other gases into the patient's hypopharyngeal space. The airway device may include one or more suction channels configured to enable removal of the patient's airway secretions. The airway device may include one or more channels configured to enable delivery of various anesthetic or other medications to enhance the placement of the device into the patient's upper airway. The channel for delivering the anesthetic agent(s) may branch such that the hypopharyngeal end contains one, two, or more than two out ports. The hypopharyngeal end of the channel(s) can be manufactured to expel the anesthetic drug in mist form. The airway device may include optics and/or one or more channels for transmission of video images seen at the hypopharyngeal end of the device. The airway device may include a pCO2 (or other partial pressure gas) detector at various positions of the device, including the hypopharyngeal end, and/or one or more channels for transmission of the measurement to the mouth end. The airway device may include a wire and channel system that is arranged to change the radius of the airway device so as to optimize positioning of the distal end of the main body in the patient's upper airway. Any one or more of the channels noted above may be of various sizes and may be disposed on the inner wall surface, the outer wall surface, or within the wall of the main tubular body of the airway device. Any one or more of the channels noted above can be sized and shaped and disposed such that the mouth ends, or proximal ends, of the channels connect easily and securely to industry standard tubing and syringes. The airway device may include one or more break-away features disposed in or along the wall or walls of the tubular body and arranged such that after placement of the airway device and an endotracheal tube or other medical device into a patient's airway through the airway device, the walls of the tubular body of the airway device separate to open at one or more radial sections through the walls of the airway device, enabling the removal of the airway device without removing the endotracheal tube or other medical device. The material of the airway device may be composed of two or more substances along the device's length so as to enhance its functionality. The mouth end, or proximal end, of the airway device may be manufactured with a substance resistant to biting. The airway device may be impregnated with various drugs, such as for example anesthetic agents, to enhance placement into the patient's upper airway. The airway device may include an inflatable cuff along its hypopharyngeal aspect. The distal, or far, end of the airway device may have a length selected so that, when inserted from the mouth of a patient, the distal end will be disposed in the hypopharyngeal space and the proximal end will extend through the mouth of the patient. However, the airway device may be sized with a length so as to end at any location from the oral cavity to the hypopharyngeal space close to the vocal cords when inserted from the mouth of a patient. The airway device may contain any number and combination of the features and arrangements noted above.

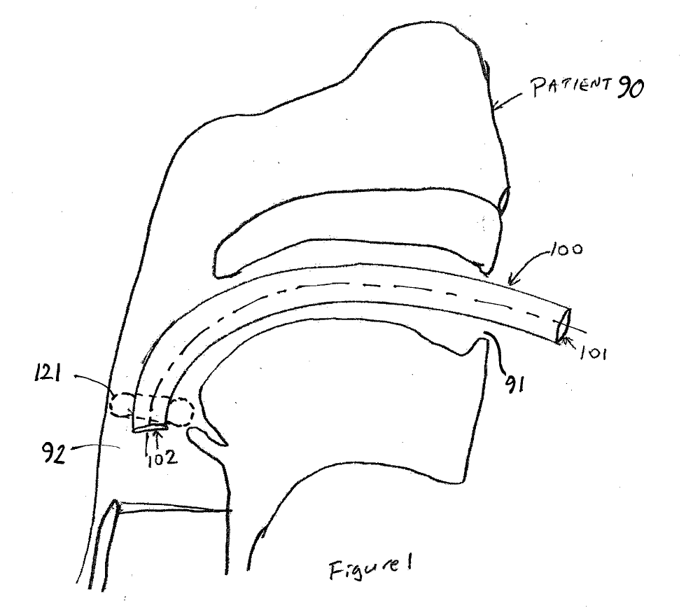

Turning now to the exemplary arrangements of the drawings, FIGS. 1 through 4 illustrate a first embodiment of an airway device of the present invention. FIG. 1 illustrates the airway device 100 inside a cross sectional view of a patient 90. The airway device 100 has a main body with the general shape of an elongate tube formed by a tube wall extending along a longitudinal axis between a proximal or mouth end 101 and a distal or hypopharyngeal end 102. When inserted into an operative position in the patient, the or mouth end, or proximal end, 101 of the airway device 100 protrudes outside of the patient's mouth 91 sufficiently such that the device will be able to be manipulated, such as by the fingers of a medical professional, to ensure a good fit and provide access to various working ports that are optionally incorporated into the airway device. The hypopharyngeal end, or distal end, 102 of the airway device 100 is designed to rest in the hypopharyngeal space 92 of the patient 90 as shown in FIG. 1 when the proximal end 101 protrudes out of the patient's mouth 91 as described previously. This enables access to the patient's glottis (i.e., the opening through vocal cords) with various medical devices. The tube wall of the main body of the airway device 100 is shown to be circular in cross-sectional shape transverse to the longitudinal axis of the elongate tubular shape, thereby forming an elongate generally circular orifice or bore extending from the proximal end 101 to the distal end 102; however, the tube wall of the main body can be manufactured in other cross-sectional shapes, such as oval. Preferably, the main body has the same basic cross-sectional shape along the entire length from the proximal end 101 to the distal end 102, although some minor variations and variations in the cross-sectional shapes of additional optional features as described hereinafter may be present.

FIG. 2 illustrates the side view of the airway device 100 in one optional form. An optional suction port 103 is provided with the main body of the airway device 100. The suction port 103 extends along the length of the main body from the mouth or proximal end 101 to the hypopharyngeal or distal end 102. At the mouth end, the suction port 103 has a connector, such as a serrated tapering end 105, that enables a secure connection to suction tubing. An optional second suction port 104 is provided that also extends the length of the main body of the airway device 100. At the mouth end, the suction port 104 has connector, such as a serrated tapering end 106, that enables a secure connection to oxygen tubing. Each suction port 103 and 104 is in the form of an elongate tube that forms an enclosed channel or passage from the proximal end 101 to the distal end. As shown in FIG. 2, each suction port 103 and 104 is disposed on the outer side of the tube wall of the main body; however, either or both of the suction ports may be disposed on the inner side of the tube wall and/or totally or partially embedded within the tube wall. Both suction ports 103 and 104 can be used for multiple purposes, including suction and oxygen delivery, respectively, as well as other purposes. For instance, either passage 103 or 104 can be used to deliver anesthesia, such as lidocaine, into the oral cavity, oropharynx (the space in back of oral cavity) and/or the hypopharynx, for example sequentially as the device is moved into position and the airway mucosa (lining) is anesthetized.

In another optional arrangement, the airway device 100 may have only one (for example, the oxygen channel) or no channels, such as the suction ports 103 or 104.

The airway device 100 can be manufactured and/or provided in a variety of pre-defined lengths, diameters, and shapes so as to conform most appropriately to patients of various ages, sizes, and shapes. The contour and material of the airway device 100 can be constructed so as to optimize the patient's comfort. In some arrangements, the main body is contoured such that the distal end 101 rests directly above the glottis or tracheal opening instead resting directly above the opening into the esophagus when inserted in its operative position in the upper respiratory tract. In one arrangement as shown in the Figures, the main body of the airway device 100 is contoured to have a variable curve along its longitudinal length. The curve of the contour varies from a long radius (i.e., relatively flat) at the mouth end 101 to a short radius (i.e., relatively tight curve) at the hypopharyngeal end 102 in such a manner to more closely approximate the curvature of the patient's mouth and hypopharyngeal cavity, as shown in FIG. 1. The length of the airway device 100 can be manufactured shorter so that the distal end 102 rests in the patient's oral cavity or oropharynx when inserted in the operative position. Alternatively, the airway device 100 can be lengthened so the distal end 201 rests immediately above the vocal cords in the hypopharynx when inserted in the operative position. Different lengths of the device would function for various uses and patient comfort levels.

The oxygen 104 and suction 103 ports and channels can be manufactured with different diameters, which could enable different functionalities in various situations and patients. These channels can be located on the outside of the invention as seen in FIG. 2 or on the inner diameter of the main body of the invention as seen on cross section in FIG. 4.

The main body of the airway device 100 is preferably made of material that is semi-rigid yet flexible such that that the main body conforms to a patient's airway without much distortion of its resting form. The flexibility is preferably sufficient to enable the main body to be placed readily into the patient's airway without causing mucosal erosions. The chemical and physical composition of the airway device 100 may be composed of one or more compounds with one or more varying densities and one or more wall thicknesses. This enables different functionalities based on the position of the airway device 100. For example, the part of the airway device 100 that is adjacent to the patient's teeth at the proximal end 101 may be made more resistant to biting by being made of a more rigid material, than elsewhere along the main body of the airway device 100.

In some arrangements, the airway device 100 may be impregnated with an anesthetic agent, such as lidocaine, that will leak out of its substance and anesthetize the mucosal lining of the airway. The anesthetic agent can be impregnated at various different locations along the main body of the airway device. Other drug categories, such as sedatives and analgesics, may also or alternatively be impregnated into the airway device to enhance its effectiveness in preparing the patient's upper airway to receive the airway device.

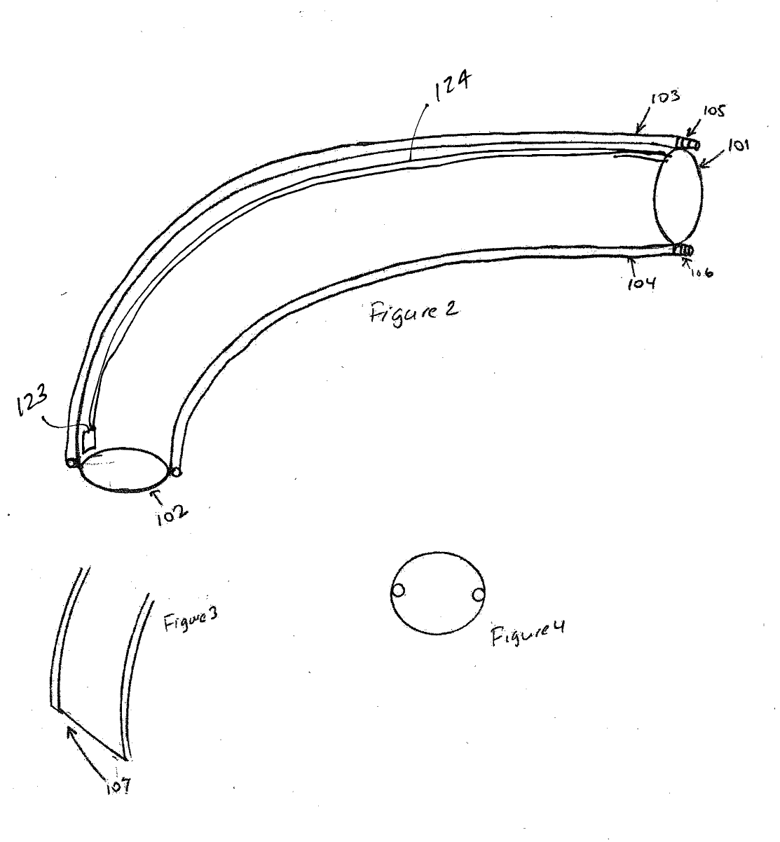

The hypopharyngeal end 102 of the airway device 100 can be shaped such that the distal end 102 is angled toward the main body of the invention 100 to varying degrees. FIG. 2 illustrates this angle at 90 degrees to the longitudinal axis of the tube wall of the main body at the distal end 102. FIG. 3 illustrates the angle 107 of the hypopharyngeal end 102 of the airway device 100 at an obtuse angle with the posterior aspect of the main body of the airway device. This angle can be manufactured at different degrees and can enable easier placement of the invention into the patient as it promotes movement of the invention along the posterior pharynx (back of oral cavity).

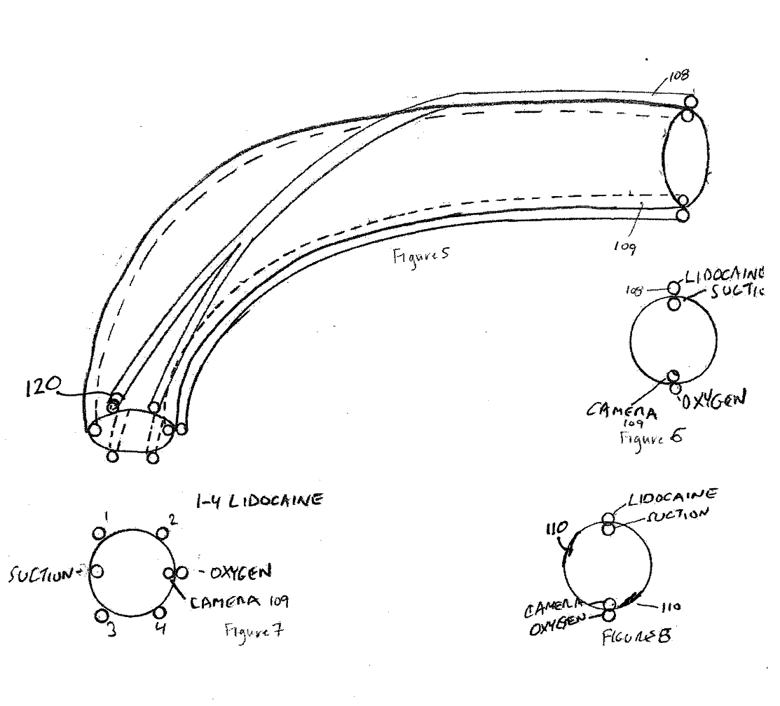

FIG. 5 illustrates the airway device 100 on lateral view with yet additional optional features. One optional arrangement includes a branching channel system 108 with one or more branching tubes that extend along the length of the main body and that dispenses topical liquid anesthetic agents, as lidocaine. This enables the airway device to be progressively and easily advanced into its final position by moving the main body forward while intermittently injecting a topical anesthetic agent. As seen in FIG. 6, the mouth end 101 of the branching channel system 108 has one orifice that will enable it be connected to standard sized and shaped syringes and tubing that are commercially available. As shown in FIG. 7, the hypopharyngeal end 102 of the branching channel system 108 can be manufactured such that it contains from one to many channels. FIGS. 5 and 7 illustrate the branching channel system containing four channels and out ports at the hypopharyngeal end; more or fewer channels may be provided.

The tip of the hypopharyngeal end 102 of one or more of the branching channels 108 can be manufactured such that an anesthetic agent egresses in a mist form, such as a with a misting or aerosolizing nozzle 120, so as to enable a more complete coverage of action for the topical anesthetic agent.

Towards the hypopharyngeal end 102 of the airway device 100, an inflatable cuff 121, as shown in dashed lines in FIG. 1, may optionally be placed such that it seals the upper airway of the patient. The cuff 121 will assist in keeping oral secretions from entering the patient's hypopharyngeal space and can be used to provide a temporary airway to ventilate a patient.

Other options for the airway device 100 include a tubular channel 109 with appropriate wires or fiber optics that transmits electronically or fiber optically the image at the hypopharyngeal end of the device. The optics for this function would be placed at the hypopharyngeal end of the airway device 100. The mouth end 101 of the tubular channel 109 can be manufactured such that it can connect to appropriate electronic or fiber optic devices enabling visualization of the hypopharyngeal end of the device in real time. The airway device 100 can also include a pCO2 (or other partial pressure gas) detector 123 positioned at various locations of the device, including the hypopharyngeal end, and transmit the reading to the mouth end 101. One or more channels 124 may extend from the detector 123 along the main body to the proximal end 101 for transmission of the measurement to the mouth end.

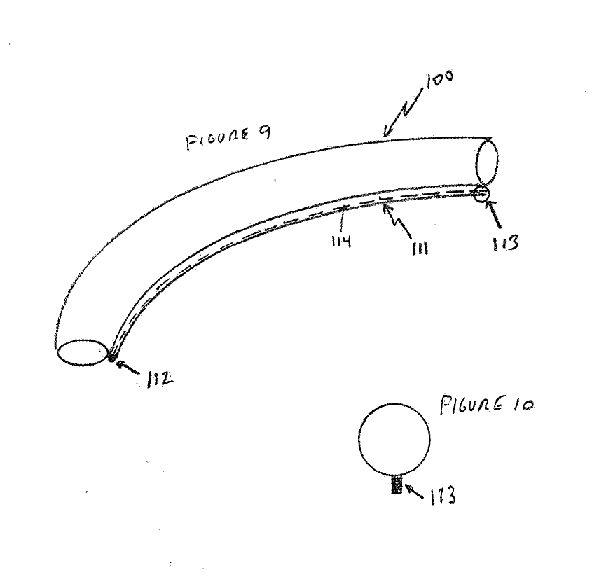

As shown in FIG. 8, the airway device 100 can include a feature to remove it after placement of an endotracheal intubation airway device. This feature entails a break away section 110 that extends linearly along the length of the device as seen in FIG. 8. The break away section 110 may be, for example, a line of weakness or perforation or a snap apart connection; however other arrangements of break away sections are also possible. In FIG. 8, this break away section 110 is shown on one part extending radially through the tube wall of the main body of the airway device 100 but can also be placed along a second position, such as radially opposite the first position, such that two parts of the main body will be separated completely when removing the airway device.

The airway device 110 can also have an adjustment mechanism that shortens and/or lengthens the radius of the ventral (chin) side of the main body, which will change the overall radius of the curve of the contour of the device 100 as shown in FIGS. 9 (side view) and 10 (cross sectional view). This will enable more precise positioning inside the patient's airway such that the hypopharyngeal end of the device can be directly positioned in front of the patient's vocal cords. The adjustment mechanism can be enabled by manufacturing a channel 111 on the ventral side of the main body of the airway device 110 that will transmit a wire 114, which when tensioned will shorten the longitudinal radius of the airway device. The wire 114 can be attached to an anchoring point 112 on the distal end of the airway device 110. To control the tension of the wire 114 and the radius of the airway device 110, a spool 113 that is arranged to selectively shorten or lengthen the wire can be located at the mouth end 101 of the airway device 110. The spool 113 may have a rotational resistance so that setting the tension of the wire 114 will be static when the spool is released. The spool 113 can be made with ridges or other such features that enhance its usability by human fingers. The channel 111, wire 114, and spool system 113 can be manufactured on either the anterior or posterior side of the airway device 110.

The airway device 110 can be manufactured to contain any of the optional features stated above in various combinations based on the needs of a patient and the desires of the clinician operating the device.

This detailed description is to be construed as examples only and does not describe every possible embodiment, as describing every possible embodiment would be impractical, if not impossible. One could implement numerous alternate embodiments, using either current technology or technology developed after the filing date of this application. Thus, while specific exemplary forms are illustrated and described herein, it is to be understood that any of the various aspects, arrangements, and/or features disclosed herein may be combined with any one or more of the other aspects, arrangements, and/or features disclosed herein in a manner that would be understood by a person of ordinary skill in view of the teachings of this disclosure.

I claim:

1. An airway device for being inserted into an operative position in the upper respiratory tract extending from a patient's mouth to the patient's hypopharyngeal space, the airway device comprising:
a main body having a hollow tubular shape forming a bore extending from a proximal end to a distal end, wherein the main body is formed of a semi-rigid material that has an at-rest contour that approximates a curve of the patient's airway.

2. The airway device of claim 1, wherein the main body is contoured to have a variable curve along its longitudinal length.

3. The airway device of claim 2, wherein the curve of the contour varies from a long radius at the proximal end to a short radius near the distal end.

4. The airway device of claim 1, wherein the main body has a length that is at least as long as the upper respiratory tract extending from the patient's mouth to the patient's hypopharyngeal space.

5. The airway device of claim 4, wherein the main body is contoured along a curve such that the distal end rest directly above the patient's glottis when inserted in the operative position in the upper respiratory tract.

6. The airway device of claim 1, further comprising:
an oxygen delivery channel arranged to provide a secure route to the patient's hypopharynx that optimizes delivery of oxygen to lungs.

7. The airway device of claim 1, further comprising:
a suction channel arranged to enable removal of upper airway secretions from the patient.

8. The airway device of claim 1, further comprising:
an anesthetic channel arranged to enable anesthetization of the upper respiratory tract during insertion of the device into the upper respiratory tract.

9. The airway device of claim 8, wherein the anesthetic channel branches along the main body to form multiple exit ports at the distal end.

10. The airway device of claim 8, further comprising:
a misting nozzle located at the distal end of the anesthetic channel that is arranged to expel the anesthetic drug in mist form.

11. The airway device of claim 1, further comprising:
a video channel arranged to provide transmission of real time view of the distal end of the to an operator.

12. The airway device of claim 1, further comprising:
a partial pressure gas measurement detector arranged to obtain measurements from the along the upper respiratory tract.

13. The airway device of claim 1, further comprising:
a break-away feature disposed in a tube wall defining the main body, wherein the break-away feature is arranged in the tube wall such that, after placement of the airway device in the upper respiratory tract and a placement of a medical device along the bore, the tube wall can be separated to open at one or more radial sections through the walls of the airway device, whereby the airway device may be removed from the upper respiratory tract without removing the medical device.

14. The airway device of claim 1, wherein the main body is composed of two or more substances along the device's length.

15. The airway device of claim 14, wherein the proximal end of the main body is composed of a substance resistant to biting.

16. The airway device of claim 1, wherein the main body is impregnated with an anesthetic agent.

17. The airway device of claim 1, further comprising:
an inflatable cuff disposed near the distal end of the main body, wherein the inflatable cuff is arranged to be inflated to seal the upper respiratory tract.

18. The airway device of claim 2, further comprising:
an adjustment mechanism that is arranged to change the radius of the curve so as to optimize positioning of the distal end of the main body in the patient's upper respiratory tract.
